(19) United States
(12) Patent Application Publication
(10) Pub. No.: US 2008/0070877 A1
(43) Pub. Date: Mar. 20, 2008

(54) Process of epilobium species for treatment of hormone balance in warm blooded animals, and method of manufacturing

(76) Inventors: Rafael Vazquez Gomez, Tijuana (MX); Robert J. Jacquet, San Diego, CA (US)

Correspondence Address: GARY L. EASTMAN, EASTMAN & ASSOCIATES, 707 BROADWAY STREET, SUITE 1800, SAN DIEGO, CA, 92101, US

(21) Appl. No.: 11/899,948

(22) Filed: Sep. 7, 2007

Related U.S. Application Data
(60) Provisional application No. 60/824,775, filed on Sep. 7, 2006.
(60) Provisional application No. 60/856,962, filed on Nov. 6, 2006.

(51) Int. Cl. A61K 31/56; A61P 15/00

(52) U.S. Cl. 514/169

(57) ABSTRACT

The present invention includes the selection of a particular species of Epilobium, and the production of an efficacious Epilobium extract. The method of extraction utilized in the present invention includes the selection and separation of parts of Epilobium Parviflorum plant to be used, excluding the use of solid stems and roots. Once selected, the plant parts are combined with a solvent and water for the extraction process. In one embodiment of the present invention, the Epilobium plant parts are combined at room temperature, such as between 71 to 81 degrees (F.) or 20 to 25 degrees (C.), with 196 proof Sugar Cane Alcohol (without denaturalization process), stirred, and allowed to rest, such as for 72 hours. Next, an additional amount of Sugar Cane Alcohol and water is added, stirred, and allowed to rest, such as for 24 hours. Next, water is added, stirred, and allowed to rest, such as for 48 hours; and this step may be repeated. Next, additional sugar cane alcohol is added for stabilization, and the mixture is allowed to rest, such as for 96 hours. The mixture is then filtered.

### RELATED APPLICATIONS

This application claims the benefit of priority to co-pending U.S. Provisional Application Ser. No. 60/824,775 entitled “Process of Epilobium Species for Treatment of Hormone Balance in Warm Blooded Animals” filed Sep. 7, 2006, and co-pending U.S. Provisional Application Ser. No. 60/856,962 entitled “Process of Epilobium Species for Treatment of Hormone Balance in Warm Blooded Animals” filed Nov. 6, 2006.

### FIELD OF THE INVENTION

The present invention relates generally to herbal extracts. The present invention is more particularly, though not exclusively, useful as an herbal extract and method of manufacturing an extract, for use in the treatment of warm blooded animals, including humans.

### BACKGROUND OF THE INVENTION

Benign Prostatic Hyperplasia (BPH) is a very common disease, affecting about 30% of men aged fifty plus, 50% of men in their sixties and as many as 90% in their seventies and eighties (Source: US Department of Health & Human Services). By 2006, approximately 115 million men in the 50+ age bracket will suffer from BPH.

Historically, treatment of BPH has been attempted using traditional pharmaceutical treatments. For instance, currently there are two main classes of drugs are prescribed for BPH: alpha-blockers and 5-alpha-reductase inhibitors.

Alpha-blockers (or alpha-1 antagonists) work by relaxing the muscles at the neck of the bladder and in the prostate. In this way they reduce the pressure on the urethra and help increase the flow of urine. They do not cure BPH but help to alleviate some of its symptoms. Around 60% of men find symptoms improve significantly after 2-3 weeks of treatment with an alpha-blocker, which are also used for hypertension. Common side-effects include tiredness, dizziness and headaches.

Major drugs in this class include:
tamsulosin—marketed by developer Astellas as Harnal, and licensor Boehringer Ingelheim as Flomax or Alna.
alfuzosin—sanofi-aventis' Xatral
doxazosin—Pfizer's Cardura
and terazosin—Abbott's Hytrin
   5-alpha-reductase inhibitors, on the other hand, inhibit production of the hormone dihydrotestosterone, produced from testosterone, which contributes to prostate enlargement. Merck & Co's Proscar (finasteride) is the main drug of this type for BPH, although it is now being challenged by a newer product, GlaxoSmithKline's Avodart (dutasteride). Proscar was first launched in the U.S. in 1998 and is also used for the treatment of male-pattern baldness. Unlike alpha blockers, 5-alpha-reductase inhibitors are able to reverse BPH to some extent and may delay the need for surgery.

Despite their possible benefits, it is important to note that there are a number of severe side effects from pharmaceutical drugs. These include the following:
sudden decrease in blood pressure could occur upon standing, resulting in rare instances of fainting
runny nose
decrease in the amount of semen
dizziness
reduced sex drive and difficulty in maintaining an erection
   In addition, it takes several months of treatment before any benefit is noticed.

In light of the above, it would be advantageous to provide a non-pharmaceutical alternative to the treatment of BPH. This alternative treatment would have little or no side effects, be generally well tolerated, relatively uncomplicated to manufacture, and reasonably cost effective.

### SUMMARY OF THE INVENTION

Epilobium is an edible plant indigenous to the Americas, Europe, Africa, West Asia and India. In traditional herbal medicine, epilobium has proven to have antibacterial, anti-inflammatory, antimicrobial and antioxidant properties. It has been used successfully for bladder health maintenance, male health maintenance, hormonal imbalances, and urinary system health. Certain species have been identified as particularly beneficial in inhibiting the enzyme 5alpha-reductase [Lesuisse, 1996] and serving as an anti-inflammatory inhibitor [Juan et al 1988].

In 2003, The Journal of Pharmacy and Pharmacology published results of a epilobium-relevant study led by Annabella Vitalone, MD, a medical advisor for Phytoceutical, Inc. The study specifically addresses the use of epilobium in treating benign prostatic hyperplasia (BPH). A noncancerous enlargement of the prostate gland, BPH is generally considered to be a normal part of the male aging process with cell proliferation traced to age-related changes in hormone balance and cell-growth factors.

The present invention includes the selection of a particular species of Epilobium, and the production of an efficacious Epilobium extract. The method of extraction utilized in the present invention includes the selection and separation of parts of Epilobium Parviflorum plant to be used, excluding the use of solid stems and roots.

Once selected, the plant parts are combined with a solvent and water for the extraction process. In one embodiment of the present invention, the Epilobium plant parts are combined at room temperature, such as between 71 to 81 degrees (F.) or 20 to 25 degrees (C.), with 196 proof Sugar Cane Alcohol (without denaturalization process), stirred, and allowed to rest, such as for 72 hours. Next, an additional amount of Sugar Cane Alcohol and water is added, stirred, and allowed to rest, such as for 24 hours. Next, water is added, stirred, and allowed to rest, such as for 48 hours; and this step may be repeated. Next, additional sugar cane alcohol is added for stabilization, and the mixture is allowed to rest, such as for 96 hours. The mixture is then filtered.

In a preferred embodiment of the method of extraction, all exposure to daylight is avoided. Once filtered, Pollen and Urtica Dioica extract are added to the final extract. Utilizing the method of the present invention, the final mixture is in the range of 37 to 38 percent alcohol.

### BRIEF DESCRIPTION OF THE DRAWING

The nature, objects, and advantages of the present invention will become more apparent to those skilled in the art after considering the following detailed description in connection with the accompanying drawings, in which like reference numerals designate like parts throughout, and wherein:

FIG. 1 is a drawing showing the chemical composition of Epilobium;

FIG. 2 is a cross-section of a human male urinary tract, showing the location of the affliction of BPH;

FIG. 3 is a flow chart showing the method of manufacturing of an extract of the present invention;

FIG. 4 is a graphic illustration of the spectral composition using thin layer chromatography of the Epilobium extract manufactured in conjunction with the method of FIG. 3;

FIG. 5 is a flow chart showing an alternative embodiment of the method of manufacturing of an extract of the present invention;

FIG. 6 is a graphic illustration of the spectral composition using thin layer chromatography of the Epilobium extract manufactured in conjunction with the method of FIG. 5;

FIG. 7 is a flow chart showing an alternative embodiment of the method of manufacturing of an extract of the present invention; and

FIG. 8 is a graphic illustration of the spectral composition using thin layer chromatography of the Epilobium extract manufactured in conjunction with the method of FIG. 7;

FIG. 9 is a flow chart showing an alternative embodiment of the method of manufacturing of an extract of the present invention;

FIG. 10 is a graphic illustration of the spectral composition using thin layer chromatography of the Epilobium extract manufactured in conjunction with the method of FIG. 9;

### DETAILED DESCRIPTION OF THE INVENTION

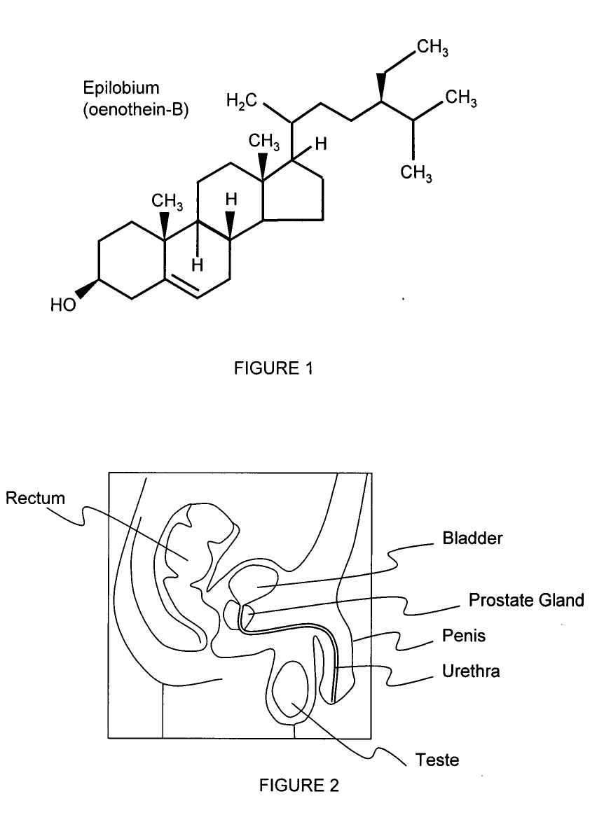

An explanation of the benefits of the use of Epilobium extracts for mammals is advantageous to the description of the present invention. The chemical composition for Epilobium is shown in FIG. 1. A clinical study was completed to determine whether various species of Epilobium, a phytotherapeutic agent, was suitable for use as a treatment for benign prostatic hyperplasia. It was identified that it may have an antiproliferative effect in PZHPV-diphenyltetrazolium bromide) test, [methyl-3H]thymidine incorporation into DNA and flow cytometry analysis were used to evaluate cell proliferation. Ethanolic extracts of E. spicatum, E. rosmarinifolium and E. tetragonum inhibited DNA synthesis in PZ-HPV-7 cells. While at high concentrations all extracts were cytotoxic, DNA synthesis was also decreased at levels that caused no or little cytotoxicity.

Treatment of cells with Epilobium extracts did not result in a formation of DNA fragments (evaluated by the TUNEL assay) or chromatin condensation (assessed by Hoechst staining). Flow cytometry analysis indicated that Epilobium extracts inhibit the progression of the cell cycle from the G0/G1 phase.

These results suggest that extracts of Epilobium inhibit proliferation of human PZ-HPV-7 cells in-vitro by affecting progression of the cell cycle.

This study provides some initial biological plausibility for the use of Epilobium extracts in benign prostatic hyperplasia (BPH). More specifically, use of an Epilobium extract has been shown to be: more cell-specific in its action; significantly more potent in BPH treatment than alternative herbal extracts, such as saw palmetto; and more powerful in blocking dihydrotestosterone (DHT), the common cause of hair loss.

Moreover, epilobium's ability to inhibit aromatase production yields a market potential that crosses gender lines. (Aromatase, an enzyme found in the liver, is key to converting androgens androstenedione and testosterone into the estrogens estrone and estradiol.) In addition to BPH management and hair growth, as discussed previously, aromatase inhibitors are currently prescribed/used to: treat clinical conditions linked with hormonal balance (i.e., estrogen and testosterone); increase lean muscle mass; and decrease body fat.

Given these factors, Epilobium is useful for the following: Prostate health in men, hair growth in adults, and menopausal management (hormonal balance) in women.

The physiology of Benign Prostatic Hyperplasia (BPH) is shown in FIG. 2. The prostate gland is approximately the size of a walnut. Located in front of the rectum and beneath the rectum, it is surrounded by fibrous tissue capsule known as the prostate capsule. The tube that transports urine and sperm—the urethra—begins at the neck of the bladder and travels through the prostate gland.

BPH rarely causes symptoms before age 40, but more than half of men in their sixties and as many as 90 percent in their seventies and eighties have some symptoms of BPH (Source: National Institutes of Health)

The precise cause of BPH—benign prostatic hyperplasia—is still being researched. It is commonly believed, however, that the levels of two hormones produced by the testes (testosterone, which is converted into dihydrotestosterone or DHT, and estradiol, or estrogen) rise as men age. This leads to growth (hyperplasia) in benign (non-cancerous) prostate cells.

As the prostate enlarges, the layer of tissue surrounding it stops it from expanding, causing the gland to press against the urethra like a clamp on a garden hose. The bladder wall becomes thicker and irritable. The bladder begins to contract even when it contains small amounts of urine, causing more frequent urination. Eventually, the bladder weakens and loses the ability to empty itself. Urine remains in the bladder. The narrowing of the urethra and partial emptying of the bladder cause many of the problems associated with BPH.

While men are affected differently, common symptoms of BPH include:
Frequent urination, particularly at night (i.e., nocturia)
Hesitant, interrupted, or weak urine stream caused by decreased force
Leakage of urine (i.e., overflow incontinence)
Pushing or straining to begin urination
Recurrent, sudden, urgent need to urinate
Blood in the urine (i.e., hematuria), caused by straining to void
Dribbling after voiding
Feeling that the bladder has not emptied completely after urination

Many people feel uncomfortable talking about the prostate, since the gland plays a role in both sex and urination. Still, prostate enlargement is as common a part of aging as gray hair. As life expectancy rises, so does the occurrence of BPH. In the United States in 2000, despite the hesitancy to seek treatment, there were 4.5 million visits to a physician for treatment of BPH.

In light of the growing incidences of of BPH, and the increasing reluctance to rely solely on pharmaceutical treatment regimens, it has been clinically clear that there are significant advantages to the of Epilobium extracts into the BPH treatment regimen.

The present invention includes the treatment of BPH using an extract of Epilobium having improved effectiveness, and the method of manufacturing that extract.

In each of the extraction processes outlined below, the process begins with the selection of various parts of Epilobium Parviflorum plant to be used, such as selection of aerial parts as leaves and new stems. It is advantageous to avoid the use of solid stems and roots. In a preferred embodiment, the Epilobium plant can be crushed as much as possible to facilitate the extraction process. It is also advantageous to maintain the temperature for the extraction process at approximately room temperature, such as between 71 to 81 degrees (F.) or 20 to 25 degrees (C.).

The process of extraction of the present invention includes a combination of the use of a Sugar Cane Alcohol, such as 196 proof and without the denaturalization process, and water. This combination of alcohol and water extraction provides for a resultant extract having a more significant level of Epilobium.

The process of extraction of the present invention includes a delay, or “resting time” in or between method steps. It is to be appreciated that this delay is exemplary of one or more preferred embodiments, and longer or shorter delays are fully contemplated herein.

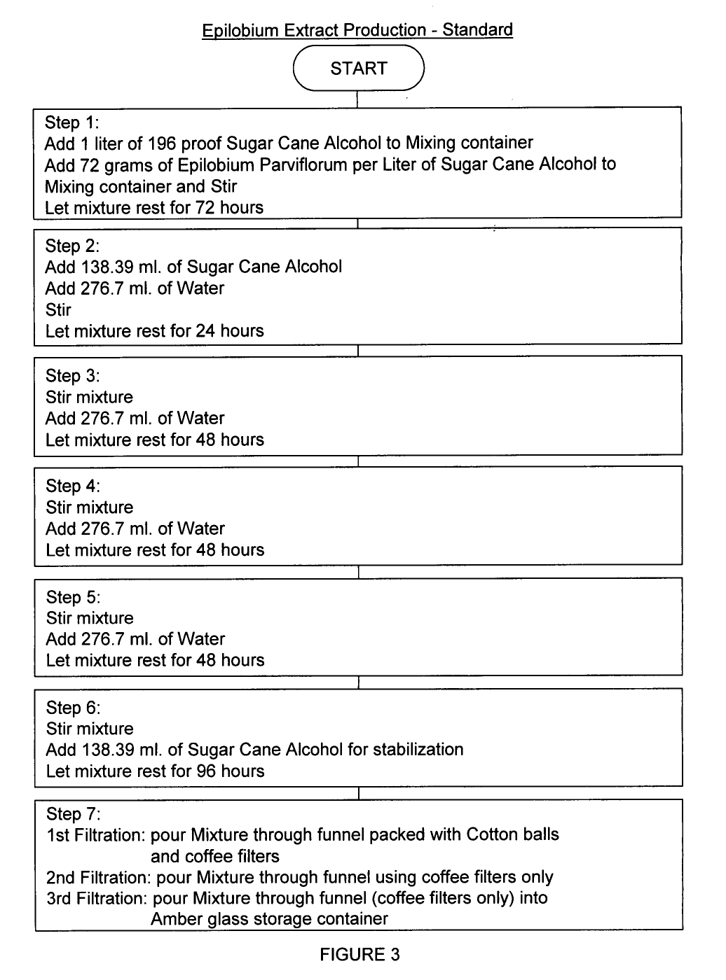

Referring now to FIG. 3, a preferred embodiment of the method of manufacturing Epilobium extracted is shown. The method of FIG. 3 includes the following method steps:

Epilobium Extract Production—Standard

Step 1:
   Add 1 liter of 196 proof Sugar Cane Alcohol to Mixing container
   Add 72 grams of Epilobium Parviflorum per Liter of Sugar Cane Alcohol to Mixing container
   Stir
   Let mixture rest for 72 hours
Step 2:
   Add 138.39 ml. of Sugar Cane Alcohol
   Add 276.7 ml. of Water
   Stir
   Let mixture rest for 24 hours
Step 3:
   Stir mixture
   Add 276.7 ml. of Water
   Let mixture rest for 48 hours
Step 4:
   Stir mixture
   Add 276.7 ml. of Water
   Let mixture rest for 48 hours
Step 5:
   Stir mixture
   Add 276.7 ml. of Water
   Let mixture rest for 48 hours
Step 6:
   Stir mixture
   Add 138.39 ml. of Sugar Cane Alcohol for stabilization
   Let mixture rest for 96 hours
Step 7:
   1^{st} Filtration: pour Mixture through funnel packed with Cotton balls and coffee filters
   2^{nd} Filtration: pour Mixture through funnel using coffee filters only
   3^{rd} Filtration: pour Mixture through funnel (coffee filters only) into Amber glass storage container
Notes:
   At any part of the process avoid exposure to daylight
   Add 3 drops (each) of Pollen and Urtica Dioica extract to the final extract
   Final mixture must be in the range of 37 to 38 percent alcohol
      Yield 2.2836 liters of extract—some evaporation during process.

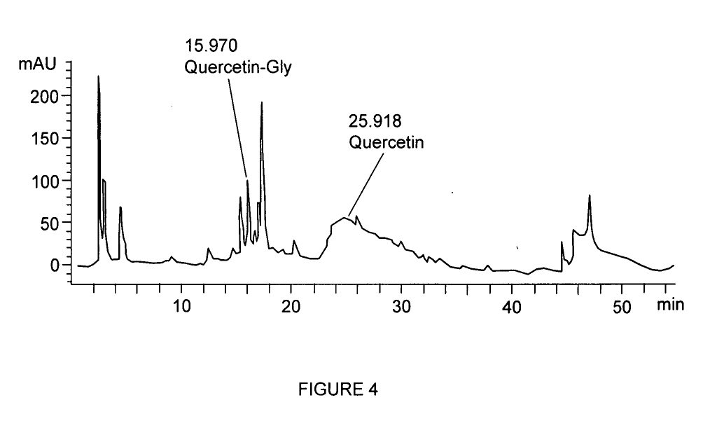

Referring now to FIG. 4, a graphic illustration of the spectral composition using thin layer chromatography of the Epilobium extract manufactured in conjunction with the method of FIG. 3 is shown. This extract of the present invention includes increased levels of flavanoids, such as Querceutin, Querceutin_Gly, and Oenothein B.

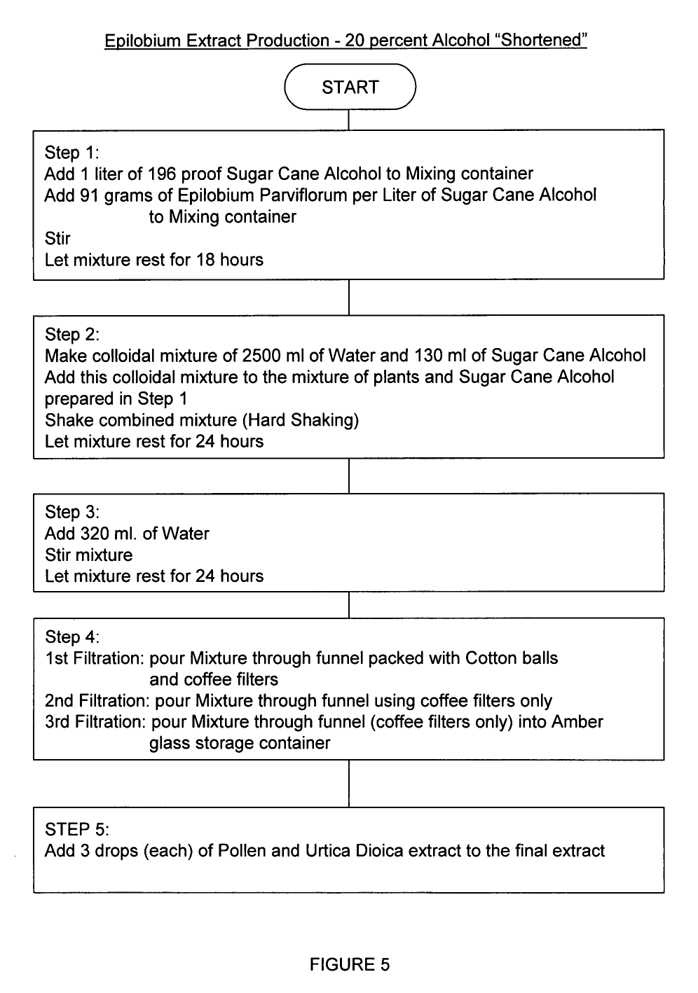

Referring now to FIG. 5, a preferred embodiment of the method of manufacturing Epilobium extracted is shown. The method of FIG. 5 includes the following method steps:

Epilobium Extract Production—20 percent Alcohol “Shortened”

Step 1:
   Add 1 liter of 196 proof Sugar Cane Alcohol to Mixing container
   Add 91 grams of Epilobium Parviflorum per Liter of Sugar Cane Alcohol to Mixing container
   Stir
   Let mixture rest for 18 hours
Step 2:
   Make colloidal mixture of 2500 ml of Water and 130 ml of Sugar Cane Alcohol
   Add this colloidal mixture to the mixture of plants and Sugar Cane Alcohol prepared in Step 1
   Shake combined mixture (Hard Shaking)
   Let mixture rest for 24 hours
Step 3:
   Add 320 ml. of Water
   Stir mixture
   Let mixture rest for 24 hours
Step 4:
   1^{st} Filtration: pour Mixture through funnel packed with Cotton balls and coffee filters
   2^{nd} Filtration: pour Mixture through funnel using coffee filters only
   3^{rd} Filtration: pour Mixture through funnel (coffee filters only) into Amber glass storage container
Notes:
   At any part of the process avoid exposure to daylight
   Final mixture must be in the range of 30±percent alcohol
   Add 3 drops (each) of Pollen and Urtica Dioica extract to the final extract
      Yield 3,425 ml of extract—some evaporation during process

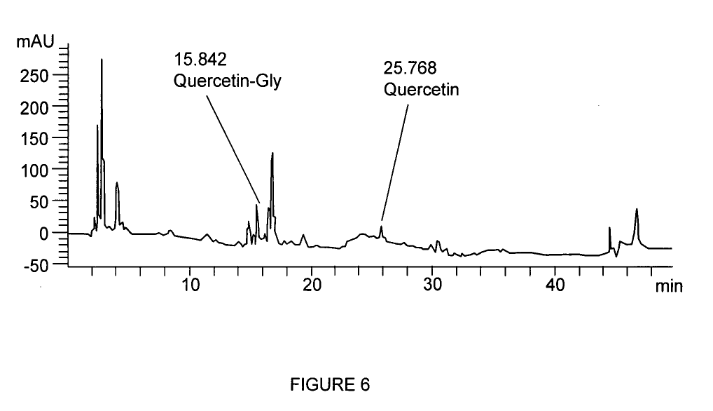

Referring now to FIG. 6, a graphic illustration of the spectral composition using thin layer chromatography of the Epilobium extract 10 manufactured in conjunction with the method of FIG. 5 is shown. This extract of the present invention includes increased levels of flavanoids, such as Querceutin, Querceutin_Gly, and Oenothein B.

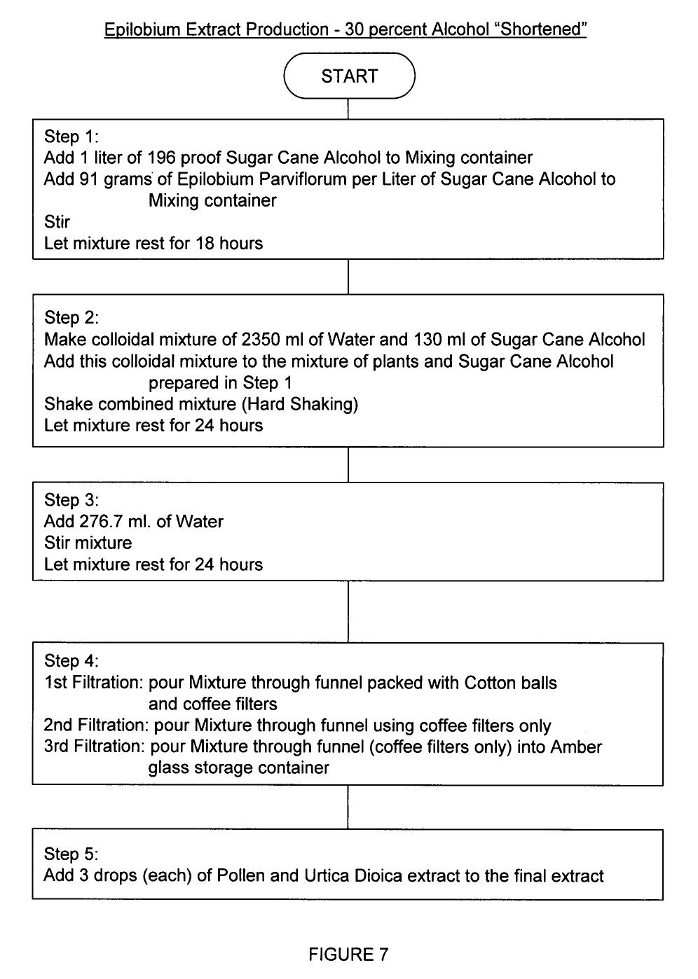

Referring now to FIG. 7, a preferred embodiment of the method of manufacturing Epilobium extracted is shown. The method of FIG. 7 includes the following method steps:

Epilobium Extract Production—30 percent Alcohol “Shortened”

Step 1:
   Add 1 liter of 196 proof Sugar Cane Alcohol to Mixing container
   Add 91 grams of Epilobium Parviflorum per Liter of Sugar Cane Alcohol to Mixing container
   Stir
   Let mixture rest for 18 hours
Step 2:
   Make colloidal mixture of 2350 ml of Water and 130 ml of Sugar Cane Alcohol
   Add this colloidal mixture to the mixture of plants and Sugar Cane Alcohol prepared in Step 1
   Shake combined mixture (Hard Shaking)
   Let mixture rest for 24 hours
Step 3:
   Add 276.7 ml. of Water
   Stir mixture
   Let mixture rest for 24 hours
Step 4:
   1^{st} Filtration: pour Mixture through funnel packed with Cotton balls and coffee filters
   2^{nd} Filtration: pour Mixture through funnel using coffee filters only
   3^{rd} Filtration: pour Mixture through funnel (coffee filters only) into Amber glass storage container
Notes:
   At any part of the process avoid exposure to daylight
   Final mixture must be in the range of 30±percent alcohol
   Add 3 drops (each) of Pollen and Urtica Dioica extract to the final extract
      Yield 3,400 ml of extract—some evaporation during process

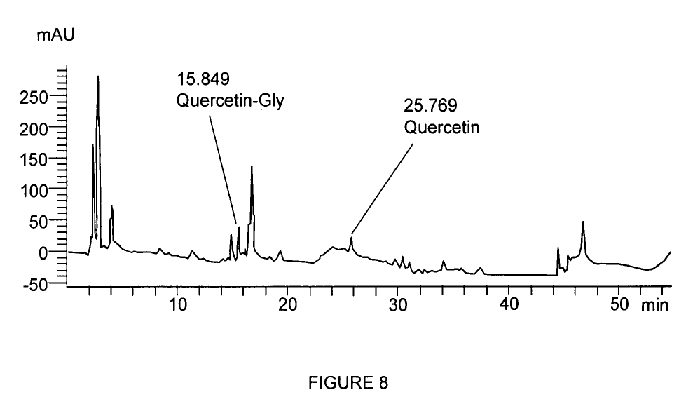

Referring now to FIG. 8, a graphic illustration of the spectral composition using thin layer chromatography of the Epilobium extract manufactured in conjunction with the method of FIG. 7 is shown. This extract of the present invention includes increased levels of flavanoids, such as Querceutin, Querceutin_Gly, and Oenothein B.

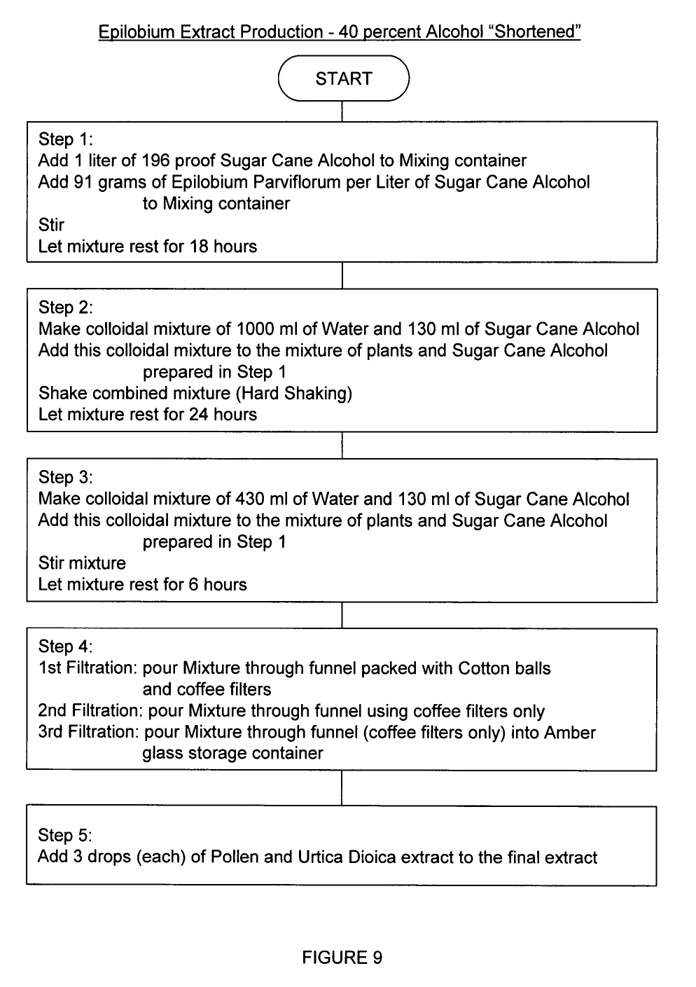

Referring now to FIG. 9, a preferred embodiment of the method of manufacturing Epilobium extracted is shown. The method of FIG. 9 includes the following method steps:

Epilobium Extract Production—40 percent Alcohol “Shortened”

Step 1:
   Add 1 liter of 196 proof Sugar Cane Alcohol to Mixing container
   Add 91 grams of Epilobium Parviflorum per Liter of Sugar Cane Alcohol to Mixing container
   Stir
   Let mixture rest for 18 hours
Step 2:
   Make colloidal mixture of 1000 ml of Water and 130 ml of Sugar Cane Alcohol
   Add this colloidal mixture to the mixture of plants and Sugar Cane Alcohol prepared in Step 1
   Shake combined mixture (Hard Shaking)
   Let mixture rest for 24 hours
Step 3:
   Make colloidal mixture of 430 ml of Water and 130 ml of Sugar Cane Alcohol
   Add this colloidal mixture to the mixture of plants and Sugar Cane Alcohol prepared in Step 1
   Stir mixture
   Let mixture rest for 6 hours
Step 4:
   1^{st} Filtration: pour Mixture through funnel packed with Cotton balls and coffee filters
   2^{nd} Filtration: pour Mixture through funnel using coffee filters only
   3^{rd} Filtration: pour Mixture through funnel (coffee filters only) into Amber glass storage container
      Notes:
   At any part of the process avoid exposure to daylight
   Final mixture must be in the range of 40±percent alcohol
   Add 3 drops (each) of Pollen and Urtica Dioica extract to the final extract
   Yield 2,350 ml of extract—some evaporation during process

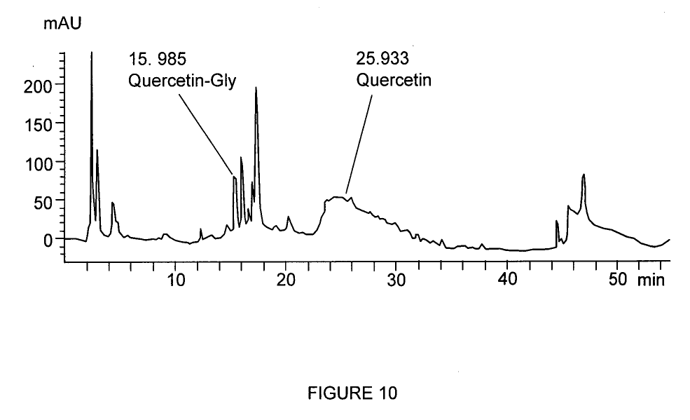

Referring now to FIG. 10, a graphic illustration of the spectral composition using thin layer chromatography of the Epilobium extract manufactured in conjunction with the method of FIG. 9 is shown. This extract of the present invention includes increased levels of flavanoids, such as Querceutin, Querceutin_Gly, and Oenothein B.

While there have been shown what are presently considered to be preferred embodiments of the present invention, it will be apparent to those skilled in the art that various changes and modifications can be made herein without departing from the scope and spirit of the invention.

1. A method of manufacturing an extract of Epilobium, comprising the steps of:
selecting parts of Epilobium Parviflorum plant;
combining said plant and Sugar Cane Alcohol to make a mixture;
stirring said mixture;
resting said mixture; and
filtering said mixture to yield an Epilobium extract.

2. The method of claim 1, further comprising the step of adding water to said mixture.

3. The method of claim 2, wherein said adding water comprises adding 276.7 ml. of Water per 72 grams of Epilobium Parviflorum per liter of sugar cane alcohol.

4. The method of claim 1, wherein said filtering step further comprises the steps of:
pouring said mixture through a funnel packed with Cotton balls and coffee filters;
pouring said mixture through a funnel using coffee filters only; and
pouring said mixture through a funnel using coffee filters only.

5. The method of claim 1, further comprising the step of adding additional Sugar Cane Alcohol for stabilization.

6. The method of claim 1, wherein said sugar cane alcohol is 196 proof Sugar Cane Alcohol.

7. The method of claim 1, wherein said parts of Epilobium plant comprises 72 grams of Epilobium Parviflorum per liter of sugar cane alcohol.

8. The method of claim 1, wherein said resting step includes a delay of about 72 hours.

9. The method of claim 1, further comprising the step of adding additional Sugar Cane Alcohol to said mixture.

10. The method of claim 1, wherein said stirring step includes mixing the mixture.

11. The method of claim 1, further comprising adding 3 drops of Pollen extract to said mixture.

12. The method of claim 1, further comprising adding 3 drops of Urtica Dioica extract to said mixture.

13. The method of claim 1, further comprising the step of avoiding exposing said mixture to daylight.

14. The method of claim 1, further comprising the step of crushing said plant.

15. The method of claim 1, further comprising the step of maintaining the temperature of said mixture between 20 to 25 degrees (C.).

16. The method of claim 1, further comprising the step of making said mixture a colloidal mixture of 2500 ml of Water and 130 ml of Sugar Cane Alcohol.

17. A method of manufacturing an extract of Epilobium, comprising the steps of:
selecting a weight of Epilobium Parviflorum plant;
combining said plant and a volume of sugar cane alcohol to make a mixture;
stirring said mixture;
resting said mixture; and
filtering said mixture to yield an Epilobium extract.

18. The method of claim 17, wherein said weight of plant is 91 grams of Epilobium Parviflorum per Liter of Sugar Cane Alcohol.

19. A method of treating benign prostatic hyperplasia (BPH), comprising the steps of administering a volume an Epilobium extract mixture made using the method of manufacturing comprising the steps of:
selecting a weight of Epilobium Parviflorum plant;
combining said plant and a volume of sugar cane alcohol to make a mixture;
stirring said mixture;
resting said mixture; and
filtering said mixture to yield an Epilobium extract.

20. The method of claim 19, wherein said volume is 500 mg per day.
